(19) United States
(12) Patent Application Publication
(10) Pub. No.: US 2006/0280814 A1
(43) Pub. Date: Dec. 14, 2006

(54) Nutritional composition for increasing creatine uptake in skeletal muscle

(76) Inventors: Paul T. Gardiner, Mississauga (CA); Marvin A. Heuer, Etobicoke (CA)

Correspondence Address: KENYON & KENYON LLP, ONE BROADWAY, NEW YORK, NY, 10004, US

(21) Appl. No.: 11/506,282

(22) Filed: Aug. 17, 2006

Related U.S. Application Data
(62) Division of application No. 11/122,523, filed on May 4, 2005.
(60) Provisional application No. 60/569,049, filed on May 7, 2004.
(60) Provisional application No. 60/580,114, filed on Jun. 15, 2004.

(51) Int. Cl. A61K 31/522; A61K 31/205; A61K 36/82; A61K 36/54

(52) U.S. Cl. 424/729; 424/739; 424/771; 514/263.31; 514/554; 514/290

(57) ABSTRACT

A nutritional composition, the consumption of which provides a method for increasing creatine accumulation, building muscle size, increasing thermogenesis, reducing body fat mass leading to weight loss and/or improving muscular definition. The nutritional composition may include an aqueous solution of cinnamon and creatine. In addition, the nutritional composition may also include alpha lipoic acid. A method of manufacturing the nutritional composition is also provided.

### RELATED APPLICATIONS

This application is related to U.S. Provisional Patent Application Ser. No. 60/569,049, filed on May 7, 2004, and U.S. Provisional Patent Application Ser. No. 60/580,114, filed on Jun. 15, 2004, each of which is hereby incorporated by reference in its entirety.

### FIELD OF THE INVENTION

The present invention relates to the retention of creatine within the body, and relates in particular but not exclusively to methods and compositions for increasing creatine accumulation in humans for the purpose of, e.g., building muscle size. In addition or alternatively, the present invention may also provide methods and compositions for increasing thermogenesis in an animal, for the purposes of, e.g., reducing body fat mass leading to weight loss and improving muscular definition.

### BACKGROUND

Recent in vitro experiments have shown that polyphenolic polymers contained in aqueous extracts from cinnamon (i.e. Cinnamomum varieties) improve cellular glucose metabolism. By promoting phosphorylation of the insulin receptor and by inhibiting the dephosphorylation of the insulin receptor kinase, such extracts have been demonstrated to trigger the insulin cascade system and potentiate the activity of insulin, thereby increasing insulin sensitivity and stimulating glucose uptake and glycogen synthesis.

The newly characterized chemical structures are closely related to previously reported derivatives of cinnamon, MHCP—methylhydroxychalcone polymers. Chemically speaking, these polyphenolic polymers are doubly linked type-A procyanidin oligomers of catechins/epicatechins.

A series of in vivo studies in animals have demonstrated that cinnamon extracts (consumed either at 2% of total diet or in amounts ranging from 30 to 300 mg/kg/day) are able to dose-dependently ameliorate plasma glucose, insulin and triglycerides levels, and increase glucose uptake in skeletal muscle—at least in part through enhancing insulin signalling (i.e., increased levels of IR-β activation and IRS-1 tyrosine phosphorylation, added to higher IRS-1/PI 3-kinase association) and via activation of the nitric oxide (NO) pathway.

Also human trials have shown positive influences of cinnamon supplementation on glucose and lipid metabolism. In one study, a single dose of dietary cinnamon (55 mg/kg b.w.) significantly blunted (P=0.02) the glycemic response to a 75 g-glucose challenge in 6 healthy female subjects. The area under the glucose curve decreased with cinnamon consumption, possibly by enhancement of insulin activity.

A recent placebo-controlled study conducted in type-2 diabetics showed that modest daily intake of table cinnamon (i.e., 1 to 6 g per day consumed, in 500-mg capsules, immediately after the main meals for 40 consecutive days) was able to safely reduce mean fasting serum glucose, triglyceride, LDL cholesterol, and total cholesterol levels (while no changes were noted in the subjects of the placebo groups).

Interestingly enough, the study also reported the maintenance of lower serum glucose and lipid levels when the individuals stopped taking cinnamon for 20 days, suggesting that cinnamon would not need to be consumed every day. According to the investigators, the main components responsible for the hypoglycemic action of cinnamon bark are the water soluble polyphenolic polymers, which appear to be non-toxic in any quantity (as opposed to fat-soluble compounds from cinnamon, which may accumulate in the body if ingested over a long period of time). Also, the levels of cinnamon tested in this study suggest that there is a wide range of cinnamon intake that may be beneficial and that intake of <1 g daily is likely to be beneficial in controlling blood glucose and lipid levels.

Extracts of cinnamon have been shown (in vitro) to activate glycogen synthase, increase glucose uptake, and inhibit glycogen synthase kinase-3β. Extracts of cinnamon have also been shown to activate insulin receptor kinase and inhibit dephosphorylation of the insulin receptor. All of these effects would lead to increased insulin sensitivity.

An additional mechanism of action for improved cellular glucose uptake following consumption of cinnamon extracts seems to reside in the effect that polyphenolic fractions might exert on endothelial nitric oxide (NO). Evidence exist that polyphenolic compounds are capable of inducing endothelium-dependent relaxation, and that this effect results from enhanced synthesis of NO, enhanced biological activity of NO and protection against its breakdown by O₂. Enhanced synthesis of NO and improvement of its biological activity, would ensure increased blood flow (also mediated via enhanced insulin-mediated vasodilation), thereby supporting the view that modulation of blood flow is a determinant of glucose uptake and glucose delivery to the tissues.

In addition, increased bioavailability of bradykinin can be proposed as a possible mechanism of improved cellular glucose metabolism with cinnamon extract supplementation. In fact, a recent investigation has shown that butein (i.e., 3,4,2′,4′-tetrahydroxychalcone from Rhus verniciflua, a plant extensively used in Korean folks medicine), a polyphenol similar in structure to the compounds found in cinnamon aqueous extract, has hypotensive effects via the inhibition of angiotensin converting enzyme (ACE). The inhibition is likely to be mediated via the generation of chelate complexes with zinc ions within the active center of ACE, thus inactivating the ACE activity.

Recent human studies have demonstrated that ACE inhibition improves glucose disposal rate and that the effect may be primarily due to increased muscle glucose uptake (MGU). In insulin-resistant conditions, ACE inhibitors can also enhance whole-body glucose disposal and glucose transport activity in skeletal muscle. The hemodynamic effects of ACE inhibition are associated with enhanced levels of the vasodilator peptide bradykinin (BK) and decreased production of the vasoconstrictor and growth factor angiotensin II (ATII). These results are not surprising because ACE, which is identical to the BK-degrading kininase II, is abundantly present in muscle tissue, and its inhibition has been shown to elicit the observed metabolic actions via elevated tissue concentrations of BK and through a BK receptor site (B₂) in muscle and/or endothelial tissue.

Exogenous BK applied into the brachial artery of the human forearm not only augmented muscle blood flow (MBF) but also enhanced the rate of MGU. In another investigation, during rhythmic voluntary contraction, both MBF and MGU increased in response to the higher energy expenditure, and the release of BK rose in the blood vessel, draining the working muscle tissue.

At the cellular level, ACE inhibitors acutely enhance glucose uptake in insulin-resistant skeletal muscle via two mechanisms. One mechanism involves the action of bradykinin, acting through bradykinin B₂ receptors, to increase NO production and ultimately enhance glucose transport. A second mechanism involves diminution of the inhibitory effects of ATII, acting through angiotensin receptors (AT₁), on the skeletal muscle glucose transport system.

The acute actions of ACE inhibitors on skeletal muscle glucose transport are associated with upregulation of insulin signaling, including enhanced IRS-1 tyrosine phosphorylation and phosphatidylinositol-3-kinase activity, and ultimately with increased cell-surface GLUT-4 glucose transporter protein. Chronic administration of ACE inhibitors or AT₁ antagonists to insulin-resistant rodents can increase protein expression of GLUT-4 in skeletal muscle and myocardium.

These data support the concept that ACE inhibitors can beneficially modulate glucose control in insulin-resistant states, possibly through a NO-dependent effect of bradykinin and/or antagonism of ATII action on skeletal muscle.

This is of interest because, in recent studies, insulin has been suggested to elicit its actions on MBF and MGU via the accelerated release of endothelium-derived nitric oxide, the generation of which is also stimulated by BK in a concentration-dependent manner. Since bradykinin is also a substrate for ACE, it might be possible that ACE inhibition by cinnamon hydroxychalcones could also result in increased bradykinin bioavailability, with consequent enhancement of GLUT4 translocating capacity and increased glucose uptake in skeletal muscle tissue.

### SUMMARY OF THE INVENTION

The present invention provides for a nutritional supplement for an animal, e.g., a human, that provides musclebuilding and/or thermogenic properties. In a preferred embodiment, the nutritional composition includes an aqueous extract of cinnamon and creatine. In one such embodiment, the creatine is provided in the form of di-creatine malate. In addition, the nutritional supplement may include alpha lipoic acid, among other ingredients, as set forth below.

The present invention also provides methods and compositions for supplementing the diet of an animal, comprising administering to the animal a serving of a nutritional supplement that provides musclebuilding and/or thermogenic properties. In a preferred embodiment, the present invention provides methods and compositions for supplementing the diet of an animal, comprising administering to the animal a serving of a nutritional composition that includes an aqueous extract of cinnamon and creatine, and which may also include alpha lipoic acid among other ingredients.

The present invention also provides methods and compositions for increasing creatine accumulation in skeletal muscle of an animal, for the purpose of, e.g., building muscle size. In addition or alternatively, the present invention may also provide methods and compositions for increasing thermogenesis in an animal, for the purposes of, e.g., reducing body fat mass leading to weight loss and improving muscular definition.

According to one embodiment of the present invention, the method comprises the steps of:

a. administering a nutritional supplement comprising a serving of creatine and an aqueous extract of cinnamon, and;

b. increasing the total muscle creatine in the skeletal muscle of an animal.

The present invention also provides methods for manufacturing a nutritional supplement. According to one embodiment of the present invention, there is provided a method of manufacturing a nutritional supplement that includes creatine, alpha lipoic acid and/or an aqueous extract of cinnamon. In one embodiment; the method includes the following steps:

a. premixing a microcrystalline cellulose with creatine, lipoic acid, and an aqueous extract of cinnamon;

b. adding magnesium stearate and silica which had been pre-sifted;

c. blending and mixing for 30 minutes;

d. checking for uniformity/homogeneity and then aliquoting into a serving.

### DETAILED DESCRIPTION

The present invention provides for a nutritional supplement for an animal, e.g., a human, that provides musclebuilding and/or thermogenic properties. In a preferred embodiment, the nutritional composition includes an aqueous extract of cinnamon and creatine. In one such embodiment, the creatine is provided in the form of di-creatine malate. In addition, the nutritional supplement may include alpha lipoic acid, among other ingredients, as set forth below.

The present invention also provides methods and compositions for supplementing the diet of an animal, comprising administering to the animal a serving of a nutritional supplement that provides musclebuilding and/or thermogenic properties. In a preferred embodiment, the present invention provides methods and compositions for supplementing the diet of an animal, comprising administering to the animal a serving of a nutritional composition that includes an aqueous extract of cinnamon and creatine, and which may also include alpha lipoic acid among other ingredients.

The present invention may also provide methods and compositions for increasing creatine accumulation in skeletal muscle of an animal comprising the steps of:

a. administering a nutritional supplement comprising a serving of creatine and an aqueous extract of cinnamon, and;

b. increasing the total muscle creatine in the skeletal muscle of an animal.

It is believed that the ingestion of a creatine supplement comprising an aqueous extract of cinnamon increases creatine accumulation in skeletal muscle at a greater level than obtained when administering creatine alone. While not wishing to be bound by theory, it is believed that extracts of cinnamon promote the phosphorylation of the insulin receptor and inhibit the dephosphorylation of the insulin receptor, enhance NO synthesis and increase the bioavailability of bradykinin. All of these effects would lead to increased insulin sensitivity. The resulting increase in plasma insulin increases the activity of a sodium-dependent muscle creatine transporter. This theory is supported by the fact that insulin augments muscle creatine accumulation in humans when present at a concentration ≧100 mU/l.

As used herein, “total muscle creatine” refers to the total phosphocreatine and total free creatine in the skeletal muscle. Those of skill in the art will appreciate that the total muscle creatine store in a healthy, nonvegetarian subjects is, on average, about 124 mmol/kg dry mass (dm), but it can vary widely among individuals from about 100 to about 150 mmol/kg dm. In a preferred embodiment the ingestion of free creatine with aqueous extract of cinnamon (about 0.1 to 1 g of aqueous extract of cinnamon/5 g of creatine four times per day for 5 days) has the ability to increase total muscle creatine at least about 24 mmol/kg dm. In a more preferred embodiment the ingestion of free creatine with aqueous extract of cinnamon (about 0.1 to 1 g of aqueous extract of cinnamon/5 g of creatine four times per day for 5 days) has the ability to increase total muscle creatine about 28 mmol/kg dm. In a most preferred embodiment the ingestion of free creatine with aqueous extract of cinnamon (about 0.1 to 1 g of aqueous extract of cinnamon/5 g of creatine four times per day for 5 days) has the ability to increase total muscle creatine about 35 mmol/kg dm.

Those of skill in the art will appreciate that the increase of total muscle creatine with the supplement refers to an average increase of total muscle creatine over a statically large population and that the increase will vary between individuals. In particular individuals with some degree of insulin resistance may have a significantly lower creatine increase than the average.

Clinical determination of creatine accumulation in skeletal muscle following ingestion of the creatine composition comprising aqueous extract of cinnamon may be measured by various methods well known to those of skill in the art. For example, creatine accumulation in skeletal muscle can be measured directly by muscle biopsy.

Direct measurement of creatine accumulation in muscle may involve taking biopsy samples from a subject. Biopsy samples are preferably frozen in liquid nitrogen, freeze-dried, and stored at −80° C. for subsequent metabolite analysis. Typically, fat is removed from the freeze dried sample by extraction with petroleum ether, muscle samples dissected free from visible blood and connective tissue and then powdered. Neutralized perchloric acid extracts may then be prepared for the spectrophotometric determination of phosphocreatine and creatine. Muscle total creatine concentration may be calculated by summing phosphocreatine and free creatine concentrations.

Creatine accumulation in skeletal muscle following ingestion of the creatine composition comprising aqueous extract of cinnamon can be estimated indirectly. Subjects ingesting creatine in combination with the low calorie creatine composition of the inventions have plasma creatine concentration and urinary creatine excretion substantially decreased when compared with creatine ingestion alone, indicating that whole body creatine retention was increased.

Measurement of creatine levels in the plasma preferably involves removing venous blood from the dorsal surface of a heated hand immediately before and 20, 40, and 60 min after the ingestion of a supplement. In addition, urine may be collected before and one on the day of ingestion of a supplement. Plasma and urine creatine were measured using high performance liquid chromatography and serum insulin was measured using a radioimmunoassay technique. See for example U.S. Pat. No. 5,968,900.

Creatine

As used herein, “creatine” refers to the chemical compound N-methyl-N-guanyl glycine, CAS Registry No. 57-00-1, also known as, (α-methyl guanido) acetic acid, N-(aminoiminomethyl)-N-glycine, and methylglycocyamine, and Methylguanidoacetic acid, and N-Methyl-N-guanylglycine, whose chemical structure is shown below. As used herein, “creatine” also includes derivatives of creatine such as esters, ethyl esters, chelates, and amides, as well as other derivatives, including derivatives that become active upon metabolism. The chemical structure of creatine is as follows:

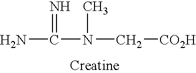

While not wishing to be bound by theory it is believed that creatine increases strength and muscle size as well as cell volumization.

Creatine and creatine derivatives are widely available from a number of commercial sources. Commercially available creatine derivatives include creatine phosphate, creatine monohydrate, creatine lactate, carnitine creatinate, creatine fumarate, creatine lipoate, creatine arginate, creatine ethyl esters, creatine anhydrous, encapsulated creatine, effervescent creatine, creatine citrate, magnesium creatine, alkaline creatine, creatine pyruvate, creatine hydrates, and tricreatine malate. Glycocyamine, and in vivo precursor of creatine, are also commercially available and suitable in the practice of the present invention.

As used herein, a serving of the supplement comprises from about 0.01 g to about 0.5 g of creatine per gram of supplement. More preferably, a serving of the supplement comprises from about 0.05 g to about 0.25 g of creatine per gram of supplement. Most preferably, a serving of the supplement comprises from about 0.1 g to about 0.2 g of creatine per gram of supplement.

In one embodiment of the present invention, the supplement comprises about 1.5 grams of dicreatine malate per serving.

Aqueous Extract of Cinnamon

As used herein, an “aqueous extract of cinnamon” preferably refers to polyphenolic polymers contained in aqueous extracts from cinnamon (i.e. Cinnamomum varieties). More preferably, “aqueous extract of cinnamon” refers to a hydroxychalcone polymer and a procyanidin type-A polymer. Most preferably, “aqueous extract of cinnamon” refers to a methylhydroxychalcone polymer and a doubly-linked type-A procyanidin oligomer of catechin/epicatechin. By promoting phosphorylation of the insulin receptor and by inhibiting the dephosphorylation of the insulin receptor kinase, such extracts have been demonstrated to trigger the insulin cascade system and potentiate the activity of insulin, thereby increasing insulin sensitivity and stimulating glucose uptake and glycogen synthesis.

Preferably, a serving of the supplement comprises from about 0.001 g to about 0.5 g of aqueous extract of cinnamon per gram of supplement. More preferably, a serving of the supplement comprises from about 0.01 g to about 0.3 g of aqueous extract of cinnamon per gram of supplement. Most preferably, a serving of the supplement comprises from about 0.02 g to about 0.2 g of aqueous extract of cinnamon per gram of supplement.

In one embodiment of the present invention, the supplement comprises about 0.025 grams of cinnamon bark extract (2% MHCP) per serving.

Alpha Lipoic Acid

As used herein, “alpha lipoic acid” preferably refers to the chemical compound 1,2-dithiolane-3-pentanoic acid, CAS registry No. 62-46-4, also known as, thioctic acid and 6,8-dithio octanoic acid, whose chemical structure is shown below. As used herein, “alpha lipoic acid” also includes derivatives of alpha lipoic acid such as esters, and amides, as well as other derivatives, such as sodium, salts of lipoic acid, creatine lipoate, R-Lipoic acid, S-Lipoic acid and including derivatives that become active upon metabolism. The chemical structure of alpha lipoic acid is as follows:

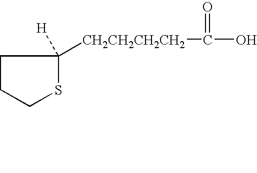

Alpha lipoic acid is an insulin modulator and an antioxidant that serves as protection against oxidative injury in non-neuronal and neuronal tissue. Alpha lipoic acid is a nutrient that the human body makes in minute quantities and may be obtained from yeast and liver. Studies have shown that alpha lipoic acid can significantly increase the body's utilization of blood sugar in type II diabetics and that lipoic acid may increase the metabolic clearance rate of glucose by 50% in diabetics. In Europe, alpha lipoic acid has been used as a substitute for insulin in the treatment of Type II diabetes.

Although the present invention is not to be limited by any theoretical explanation, it is believed that insulin is a primary factor that stimulates glucose and creatine transport into the muscle cells and that alpha lipoic acid both mimics and enhances the actions of insulin in glucose and creatine transport into the muscle cells.

Preferably, a serving of the supplement comprises from about 0.1 mg to about 100 mg of alpha lipoic acid per gram of supplement. More preferably, a serving of the supplement comprises from about 1.0 mg to about 75 mg of alpha lipoic acid per gram of supplement. Most preferably, a serving of the supplement comprises from about 25 mg to about 30 mg of alpha lipoic acid per gram of supplement.

In one embodiment of the present invention, the supplement comprises about 50 mg of alpha lipoic acid per serving.

A dosage form of the supplement may be provided as a capsule, a liquid beverage, a powder beverage mix, or as a ready-to-eat bar product. A dosage form of the supplement may be provided in accordance with customary processing techniques for herbal, dietary supplements wherein the active ingredients are suitably processed and encapsulated into cellulose capsules with suitable excipients.

Additional ingredients, which amplify creatine accumulation in skeletal muscle, may advantageously be added to the nutritional supplement. Optionally additional ingredients may be selected from the group consisting of hydroxy-isoleucine, a chromium chelate and L-taurine as well as including derivatives thereof such as esters, and amides, as well as other derivatives, including derivatives that become active upon metabolism.

For optimal effectiveness, the nutritional supplement preferably contains caffeine, catechin-polyphenols, another methyl-xanthine and combinations thereof, which further enhances creatine uptake in skeletal muscle and aids in reducing side effects.

Yerba Mate may be supplied as leaves of Ilex Paraguayensis or an enriched extract thereof. It is believed that yerba mate has several effects on the gastrointestinal system, which include prolonging the digestive period and as satiety-promoting ingredients. Preferably, a serving of the supplement comprises from about 0.1 mg to about 100 mg of yerba mate. More preferably, a serving of the supplement comprises from about 0.5 mg to about 50 mg of yerba mate. Most preferably, a serving of the supplement comprises from about 1 mg of yerba mate.

White Willow Bark (Salix Alba) is a source of acetylsalicylic acid (the major component of aspirin) which has been observed to lower serum lipoprotein (a), Lp(a), a risk factor for developing atherosclerosis. White willow bark acts on Lp(a) by reducing apolipoprotein(a), gene transcription in those patients with elevated serum lipoprotein(a). Preferably, a serving of the supplement comprises from about 0.1 mg to about 100 mg of white willow bark. More preferably, a serving of the supplement comprises from about 0.5 mg to about 50 mg of white willow bark. Most preferably, a serving of the supplement comprises from about 1 mg of white willow bark.

Huperzine is an acetyl cholinesterase inhibitor. It is believed that huperzine acts to increase growth hormone release in animals and humans. Preferably, a serving of the supplement comprises from about 0.01 mg to about 1 mg of huperzine. More preferably, a serving of the supplement comprises from about 0.02 mg to about 0.2 mg of huperzine. Most preferably, a serving of the supplement comprises from about 0.05 mg of huperzine.

Preferably, the caffeine and catechin polyphenols are supplied in combination as a tea, green tea or as an enriched tea extracts.

Preferably a serving of the nutritional supplement comprises sufficient tea, green tea or as an enriched tea extract to provide from about 25 to about 1000 mg of caffeine. More preferably, a serving of the nutritional supplement comprises sufficient green tea or enriched tea extract to provide from about 50 to about 300 mg of caffeine. Most preferably, a serving of the nutritional supplement comprises sufficient green tea or enriched tea extract to provide about 300 mg of caffeine.

Preferably a serving of the nutritional supplement comprises sufficient tea, green tea or enriched tea extract to provide from about 1 mg to about 1000 mg of a catechin-polyphenol. More preferably, a serving comprises sufficient green tea or enriched tea extract to provide from about 75 mg to about 500 mg of catechin-polyphenol. Most preferably, a serving comprises sufficient green tea or enriched tea extract to provide about 200 mg of catechin-polyphenol.

Caffeine may alternatively be supplied as essentially pure caffeine or as an ingredient naturally occurring in other ingredients. Catechin-polyphenol may also be supplied as an essentially pure catechin-polyphenol or as an enriched catechin-polyphenol. An essentially pure or enriched catechin-polyphenol may be selected from the group consisting of epigallocatechin-gallate, epicatechin-gallate, epicatechin, and epigallocatechin.

Optionally green tea is supplemented with additional tea extracts such as Oolong, black or white tea to supplement the thermogenic properties of green tea alone.

Optionally green tea is supplemented with essentially pure caffeine to supplement the thermogenic properties of green tea alone.

Optionally green tea is supplemented with an essentially pure catechin-polyphenol to supplement the thermogenic properties of green tea alone.

The nutritional supplement is preferably used to increase creatine accumulation in skeletal muscle in a person, for the purpose of, e.g., building muscle size. In addition or alternatively, the present invention may also provide methods and compositions for increasing thermogenesis in an animal, for the purposes of, e.g., reducing body fat mass leading to weight loss and improving muscular definition. Preferably the person is an athlete.

Preferably, the supplement is supplied as capsule. Alternatively, the supplement may be provided as other dosage forms, such as a tablet, caplet or as a ready-to-eat bar product. Advantageously, the supplement is consumed by a person with 8-16 ounces of water or an athletic drink.

In one embodiment a serving of the nutritional supplement is consumed by an athlete 1-4 times per day. More preferably, a serving of the supplement is administered 2 times a day.

In an alternative embodiment a serving of the supplement is administered 2 times a day 12 hours apart. More preferably, a serving of the supplement is administered 2 times a day, once in the morning and again after a workout.

In an alternative embodiment a serving of the supplement is administered 2 times a day, 12 hours apart, wherein a serving of the supplement is administered once in the morning and again before a workout.

In a further alternative embodiment the supplement is taken every day for an indefinite period of time immediately after a workout.

In an alternative embodiment the supplement is taken every day for an indefinite period of in the morning on an empty stomach.

In an alternative embodiment the supplement is taken every day for an indefinite period of in the morning and again before a workout.

In one embodiment, the present invention provides a method for manufacturing a nutritional supplement that includes creatine and an aqueous extract of cinnamon, and that may include alpha lipoic acid among other ingredients. The method may comprise the following steps:

a. premixing a microcrystalline cellulose with creatine and an aqueous extract of cinnamon;

b. adding magnesium stearate and silica which had been pre-sifted;

c. blending and mixing for 30 minutes;

d. checking for uniformity/homogeneity and then aliquoting into a serving.

Although the following examples illustrate the practice of the present invention in some of its embodiments, the examples should not be construed as limiting the scope of the invention. Other embodiments will be apparent to one skilled in the art from consideration of the specification and examples.

### EXAMPLES

### Example 1

### Dietary Supplement Content

A dietary supplement comprising the following ingredients per serving is prepared as a capsule for consumption by an athlete.

Serving Size: 3 Capsules

| | Amount Per Serving | |
|---|---|---|
| | 2.357 | |
| | g/Serving | Formula % |
| Blend 1 | 1.6 | |
| Tri-creatine malate | 1.500 | 63.6389% |
| Alpha Lipoic Acid | 0.050 | 2.1213% |
| Cinnamomum verum extract (bark) | 0.025 | 1.0606% |
| Blend 2 | .725 | |
| Green Tea Extract (leaf) | 0.444 | 18.8371% |
| Standardized for 95% polyphenols [70% | | |
| catechins (45% epigallocatechin gallate - | | |
| 175 mg EGCG)] | | |
| Caffeine (as caffeine anhydrous) | 0.250 | 10.6065% |
| Oolong tea extract (leaf) | | |
| Standardized for 50% polyphenols [25% | 0.010 | 0.4243% |
| catechins (15% epigallocatechin gallate - | | |
| 15 mg EGCG)] | | |
| Theobromine extract (as Theobrome cacao) | 0.010 | 0.4243% |
| (seed) | | |
| White tea extract (leaf) | | |
| Standardized for 50% polyphenols [35% | 0.010 | 0.4243% |
| catechins] (10% epigallocatechin gallate - | | |
| 10 mg EGCG) | | |
| Guarana extract (seed) | 0.0010 | 0.0424% |
| Yerba maté extract (as Ilex | 0.0010 | 0.0424% |
| paraguariensis) (leaf) | | |
| Standardized for 250 mg of caffeine | | |
| Blend 3 | .058 | |
| Evodia rutaecarpa extract (as | | |
| Tetradium ruticarpum) (fruit) | | |
| Standardized for 10% of evodiamine | 0.05 | 2.1213% |
| Vinpocetine | 0.0050 | 0.2121% |
| White willow extract (bark) | 0.001 | 0.0424% |
| Standardized for 25% salicin | 0.001 | 0.0424% |
| Huperzine extract (as Hyperzia serrata) (moss) | 0.00005 | 0.0021% |
| Total | 2.357 | 100% |
| Daily Value not established | | |

OTHER INGREDIENTS: Gelatin, Cellulose, magnesium stearate, silica

### Example 2

### Direction Of Use

As a dietary supplement, an individual takes 3 capsules of the dietary supplement of Example 1 with an 8 fl.-oz. glass of water 3 times daily, 60 minutes before meals. To assess individual tolerance, the dosing chart below is followed.

Dosing Chart

| | |
|---|---|
| Week 1 | 1 capsule, 3× daily |
| Week 2 | 2 capsules, 3× daily |
| Week 3 & Beyond | 3 capsules, 3× daily |

### Example 3

### Dietary Supplement Combined With Diet and Exercise

An individual combines the doses of dietary supplement determined in Example 2 with a calorie-reduced diet and a regular exercise program. The individual takes 1 of these servings, before the workout. An individual consumes ten 8 fl.-oz. glasses of water per day for general good health.

### Example 4

### Dietary Supplement

| | Serving | Active | |
|---|---|---|---|
| | 2.407 | constituent | 3 Capsule |
| Ingredients | g/serving | g/serving | Formula % |
| Dicreatine malate | 1.500 | | 62.3169% |
| Green tea dry leaf | 0.444 | 0.2 EGCG | 18.4458% |
| extract (45% EGCG, | | | |
| 75% Catechins, 90% | | | |
| Polyphenols | | | |
| Caffeine Anhydrous | 0.300 | | 12.4634% |
| White willow bark | 0.001 | 0.00025 salicin | 0.0415% |
| (25% salicin) | | | |
| Alpha lipoic acid | 0.050 | | 2.0772% |
| Cinnamon bark extract | 0.025 | 0.0005 MHCP | 1.0386% |
| (2% MHCP) | | | |
| Oolong tea dry leaf | 0.010 | 0.0015 EGCG | 0.4154% |
| extract (1 5% EGCG, | | | |
| 50% Polyphenols, 25% | | | |
| catechins) | | | |
| White tea dry leaf | 0.010 | 0.0015 EGCG | 0.4154% |
| extract (1 5% EGCG, | | | |
| 50% Polyphenols, 35% | | | |
| Catechins) | | | |
| Theobroma cocao | 0.010 | 0.0006 | 0.4154% |
| extract (6% | | theobromine | |
| theobromine) | | | |
| Evodia rutaecarpa | 0.0500 | 0.005 evodiamine | 2.0772% |
| (10% evodiamine) | | | |
| Huperzine (1% | 0.00005 | 0.0000005 | 0.0021% |
| Huperzine A) | | Huperzine A | |
| Guarana (std to 1% | 0.0010 | 0.00001 caffeine + | 0.0415% |
| caffeine + 21% | | 0.00021 from | |
| anhydrous caffeine) | | anhydrous | |
| Yerba Mate Powder | 0.0010 | | 0.0415% |
| Vinpocetine | 0.0050 | | 0.2077% |
| Total | 2.407 | | 100.0000% |

### Example 5

### Direction of Use

As a dietary supplement, an individual takes the nutritional supplement set forth in Example 4 in accordance with the following directions and warnings:

DIRECTIONS: As a dietary supplement, take 3 capsules with an 8 oz. glass of water 3 times daily, approximately 30 to 60 minutes before meals. On days of your workout, take 1 of these servings before the workout. Consume ten 8 oz. glasses of water per day. Read the entire label before use and follow directions. Do not exceed 3 capsules in a 4-hour period and/or 9 capsules in a 24-hour period. Do not take within 5 hours of bedtime. To assess individual tolerance, follow the chart.

| | |
|---|---|
| Day 1 to Day 3 | 1 capsule, 3× daily |
| Day 4 to Day 7 | 2 capsules, 3× daily |
| Day 8 & beyond | 3 capsules, 3× daily |

For best results, the nutritional composition of the present invention, and particularly the nutritional composition set forth in Example 4, is combined with an intense exercise and nutrition program.

1-24. (canceled)

25. A method for increasing creatine accumulation in an individual, the method comprising the step of:
consuming a nutritional composition comprising an extract of cinnamon and creatine.

26. The method of claim 25, wherein the extract of cinnamon is an aqueous extract of cinnamon.

27. The method of claim 25, wherein the nutritional composition further comprises alpha lipoic acid.

28. The method claim 25, wherein said creatine is provided in a form selected from the group consisting of Dicreatine Malate, Creatine Malate, Creatine Taurinate, TriCreatine Hydroxycitrate, Creatine Hydroxycitrate, Creatine Decanoate, Creatinol and derivatives thereof and Creatine Monohydrate.

29. The method of claim 25, wherein the nutritional composition is consumed in the form of a capsule.
